(19) United States
(12) Patent Application Publication
(10) Pub. No.: US 2026/0128174 A1
(43) Pub. Date: May 7, 2026

(54) Computer Application for Determining a Cardiac Score and Providing Corresponding Recommendations Via a Computing Device

(71) Applicant: Google LLC, Mountain View, CA (US)

(72) Inventors: Anthony Zahi Faranesh, Oakland, CA (US); Zeinab Esmaeilpour, San Francisco, CA (US); Davide Valeriani, London (GB); Hulya Emir-Farinas, Alameda, CA (US)

(21) Appl. No.: 18/940,249

(22) Filed: Nov. 7, 2024

(51) Int. Cl. G16H 50/30; G16H 10/60

(52) U.S. Cl. CPC G16H 50/30; G16H 10/60

(57) ABSTRACT

A computer-implemented method includes obtaining demographic data of a user. The demographic data comprises an age for the user. The method also includes obtaining physiological data of the user. The physiological data comprises one or more cardiac metrics of the user. Further, the method also includes determining, via a model, a predicted cardiac age for the user using the demographic data and the physiological data as model inputs. Moreover, the method includes determining a cardiac score based on a difference between the predicted cardiac age and the age for the user. The cardiac score is configured to assess cardiac health of the user. In addition, the method includes causing a display screen of an electronic device to display the cardiac score for the user.

### FIELD OF THE INVENTION

The present disclosure relates generally to a computer application implemented on a wearable computing device, mobile computing device, and/or server system that generates a cardiac score and provides recommendations to a user relating to the cardiac score.

### BACKGROUND

Individuals are unique and their motivational and adherence patterns in striving for a behavioral goal can vary significantly. Health-related changes in response to a behavioral change can also vary between people. Advances in sensors and wearable technologies have made it increasingly possible for individuals to collect data about themselves with the goal of self-knowledge through personal data. However, gaining self-knowledge can be more challenging than only a simple task of data collection.

For example, human cardiac health may be influenced by a plurality of factors. As such, cardiac health management may be delayed until after an onset of noticeable symptoms and/or reaching a certain age. Due to this reactive approach, individuals may lack knowledge and motivation to adopt and/or maintain behaviors associated with better cardiac health.

Accordingly, the present disclosure is directed to a computer application that can be implemented on a wearable computing device, mobile computing device, and/or server system to allow a user to proactively receive a cardiac score configured to assess cardiac health of the user.

### SUMMARY OF THE INVENTION

Aspects and advantages of embodiments of the present disclosure will be set forth in part in the following description, or can be learned from the description, or can be learned through practice of the embodiments.

In an aspect, the present disclosure is directed to a computing device having one or more processors and one or more computer-readable media that collectively store instructions that, when executed by the one or more processors, cause the computing device to perform operations. The operations include obtaining demographic data of a user, the demographic data comprises an age for the user; obtaining physiological data of the user, the physiological data comprises one or more cardiac metrics of the user; determining, via a model, a predicted cardiac age for the user using the demographic data and the physiological data as model inputs; determining a cardiac score based on a difference between the predicted cardiac age and the age for the user, the cardiac score is configured to assess cardiac health of the user; and causing a display screen of an electronic device to display the cardiac score for the user.

In another aspect, the present disclosure is directed to a computer-implemented method that includes obtaining demographic data of a user, the demographic data comprises an age for the user; obtaining physiological data of the user, the physiological data comprises one or more cardiac metrics of the user; determining, via a model, a predicted cardiac age for the user using the demographic data and the physiological data as model inputs; determining a cardiac score based on a difference between the predicted cardiac age and the age for the user, the cardiac score is configured to assess cardiac health of the user; and causing a display screen of an electronic device to display the cardiac score for the user.

In another aspect, the present disclosure is directed to a computer-implemented method that includes obtaining demographic data of a user, the demographic data comprising an age for the user; obtaining physiological data of the user, the physiological data comprising a resting heart rate, an average heart rate, a maximum heart rate, a heart rate recovery, and a maximal oxygen consumption; determining, via a model, a predicted cardiac age for the user using the demographic data and the physiological data as model inputs; determining a cardiac score based on a difference between the predicted cardiac age and the age for the user, the cardiac score configured to assess cardiac health of the user; and causing a display screen of an electronic device to display the cardiac score for the user.

These and other features, aspects, and advantages of various embodiments of the present disclosure will become better understood with reference to the following description and appended claims. The accompanying drawings, which are incorporated in and constitute a part of this specification, illustrate example embodiments of the present disclosure and, together with the description, serve to explain the related principles.

### BRIEF DESCRIPTION OF THE DRAWINGS

Detailed discussion of embodiments directed to one of ordinary skill in the art is set forth in the specification, which makes reference to the appended figures, in which:

FIGS. 1, 2, and 3 illustrate various perspective views of an example wearable computing device according to one or more example embodiments of the present disclosure.

FIG. 4 illustrates a block diagram of an example device according to one or more example embodiments of the present disclosure.

FIG. 5 illustrates a diagram of an example user assessment management system according to one or more example embodiments of the present disclosure.

FIG. 6 illustrates a diagram of an example server system according to one or more example embodiments of the present disclosure.

FIGS. 7A-7E illustrate example prompts of a cardiac health program implemented on a wearable computing device, a mobile computing device, and/or server system according to one or more example embodiments of the present disclosure, particularly illustrating a cardiac score that can be displayed by the cardiac health program.

FIG. 8 illustrates a schematic diagram of an example cardiac scoring operation according to one or more example embodiments of the present disclosure.

FIG. 9 illustrates a schematic diagram of a linear regression model that can be utilized according to example embodiments of the present disclosure.

FIGS. 10A-10B illustrate block diagrams of example machine-learned models according to example embodiments of the present disclosure.

FIG. 11 illustrates an example of a flow diagram of a computer-implemented method according to one or more example embodiments of the present disclosure.

### DETAILED DESCRIPTION

### Overview

Repeated use of reference characters and/or numerals in the present specification and/or figures is intended to represent the same or analogous features, elements, or operations of the present disclosure. Repeated description of reference characters and/or numerals that are repeated in the present specification is omitted for brevity.

As referred to herein, the terms “includes” and “including” are intended to be inclusive in a manner similar to the term “comprising.” As referenced herein, the terms “or” and “and/or” are generally intended to be inclusive, that is (i.e.), “A or B” or “A and/or B” are each intended to mean “A or B or both.” As referred to herein, the terms “first,” “second,” “third,” and so on, can be used interchangeably to distinguish one component or entity from another and are not intended to signify location, functionality, or importance of the individual components or entities. As referenced herein, the terms “couple,” “couples,” “coupled,” and/or “coupling” refer to chemical coupling (e.g., chemical bonding), communicative coupling, electrical and/or electromagnetic coupling (e.g., capacitive coupling, inductive coupling, direct and/or connected coupling, etc.), mechanical coupling, operative coupling, optical coupling, and/or physical coupling.

As referenced herein, the term “system” can refer to hardware (e.g., application specific hardware), computer logic that executes on a general-purpose processor (e.g., a central processing unit (CPU)), and/or some combination thereof. In some embodiments, a “system” described herein can be implemented in hardware, application specific circuits, firmware, and/or software controlling a general-purpose processor. In some embodiments, a “system” described herein can be implemented as program code files stored on a storage device, loaded into a memory, and executed by a processor, and/or can be provided from computer program products, for example, computer-executable instructions that are stored in a tangible computer-readable storage medium (e.g., random-access memory (RAM), hard disk, optical media, magnetic media).

As mentioned, individuals are unique and their motivational and adherence patterns in striving for a behavior goal can differ significantly. Health-related changes in response to a behavior change can also vary between people. Advances in sensors and wearable technologies have made it increasingly possible for individuals to collect data about themselves with the goal of self-knowledge through personal data. However, gaining self-knowledge can be more challenging than only a simple task of data collection.

For example, human cardiac health may be influenced by a plurality of factors. As such, cardiac health management may be delayed until after onset of noticeable symptoms or reaching a certain age. Due to this reactive approach, individuals may lack knowledge and motivation to adopt and/or maintain behaviors associated with better cardiac health.

Therefore, providing context to cardiac health can help individuals to understand its change in response to various cardiac metrics and the relationship therebetween. Moreover, understanding relationships between various cardiac metrics and corresponding behaviors can provide valuable context into interpreting the cardiac metrics measured by wearables and changes in cardiac health associated with a behavior.

Motivated by these gaps in understanding personal data, the present disclosure is directed to a cardiac health program for a computer application that is configured to proactively assess cardiac health. Thus, in an embodiment, the computer application of the present disclosure is configured to support individuals in understanding their cardiac health so as to encourage individuals to adjust their behaviors to mitigate risks of developing serious cardiac health conditions. In particular, the computer application of the present disclosure enables participants to receive a cardiac score configured to assess cardiac health of the user and track changes to their cardiac score over time in addition to rigorous insights, contextual information, and data visualizations to help participants understand their cardiac health.

According to example embodiments of the present disclosure, a computing device (e.g., a server system, a client computing device, a computer, a laptop, a tablet, a smartphone, a physiological monitoring device, a wearable computing device, a wearable physiological monitoring device (e.g., a wrist-worn device, a chest strap device)) can obtain demographic data for a user. The demographic data can include an age for the user. In one or more embodiments, the demographic data can be received via one or more human-machine interfaces (HMI) of the computing device. As such, the HMI can enable a user to interact with the computing device. In some embodiments, the HMI can include one or more interfaces that display information to a user, such as a display screen, and can also include one or more interfaces that allow a user to interact with information displayed on the screen, such as including a touch-screen component, a mouse component, a keyboard component, a stylus component, and the like. In some embodiments, the HMI can receive information from a user. For example, the HMI can receive user inputs (e.g., via sensors detecting a user pressing a virtual button on a touchscreen, via the mouse component receiving a user input specifying selection of information displayed on the screen, via the keyboard component receiving a user input specifying alphanumeric information, etc.) specifying information, such as demographic data, to the computing device.

Further, the computing device can obtain physiological data for the user. The physiological data includes one or more cardiac metrics for the user. The cardiac metric(s) may be included in heart rate (HR) data. In one or more embodiments, the HR data can be captured by one or more sensors (e.g., physiological sensors) of the computing device. As such, the computing device can obtain such HR data from such a wearable physiological monitoring device and/or another physiological monitoring device by using, for instance, a network (e.g., the Internet) as described in example embodiments of the present disclosure.

Moreover, the computing device can determine, via a model, a predicted cardiac age for the user using the demographic data and the physiological data as model inputs. Furthermore, the computing device can determine a cardiac score based on a difference between the predicted cardiac age and the age for the user. The cardiac score is configured to assess cardiac health of the user. In addition, the computing device can cause a display screen to display the cardiac score for the user. In some embodiments, the computing device includes the display screen (e.g., in the HMI). In other embodiments, the display screen may be included in a mobile computing device (e.g., that is separate and distinct from the computing device).

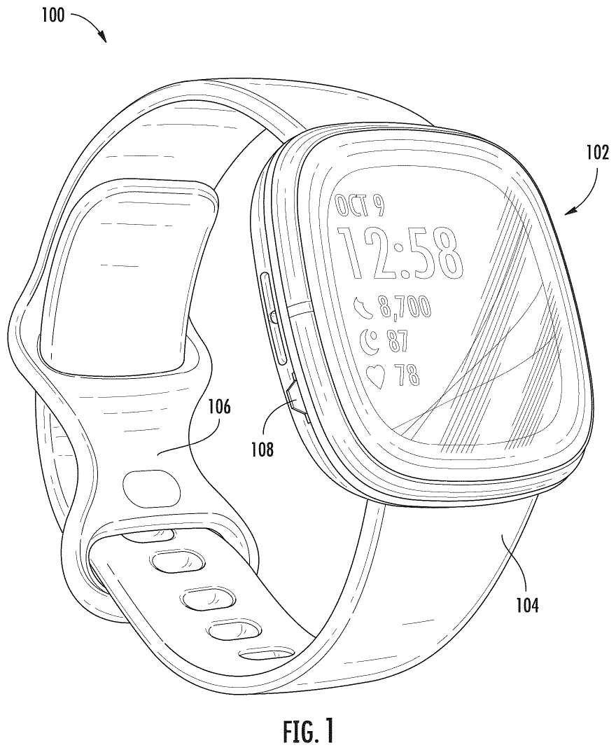

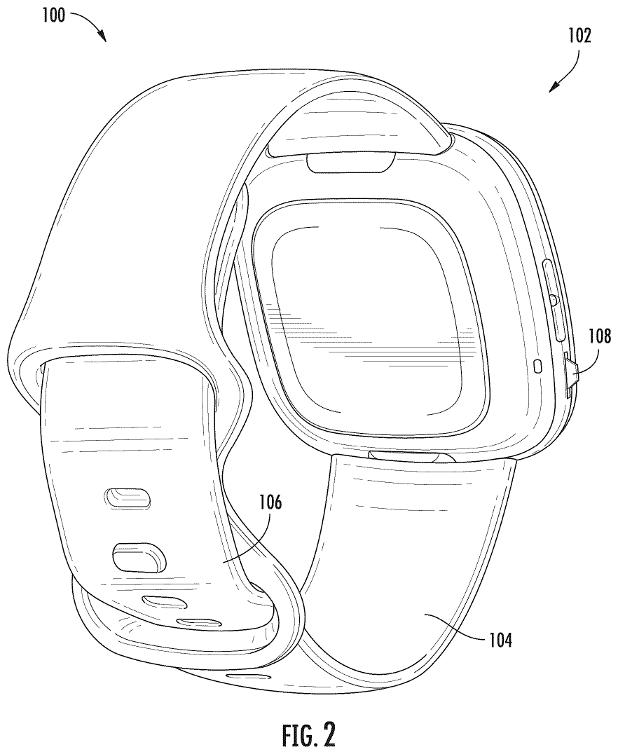

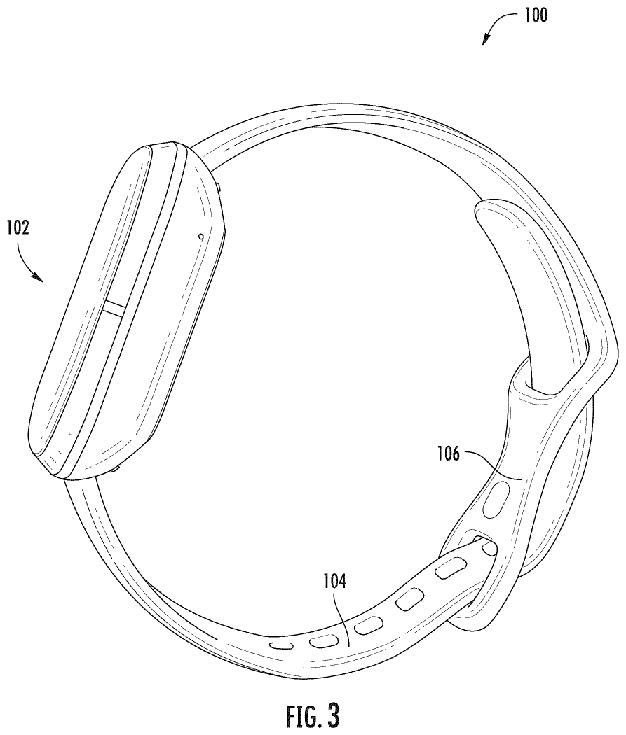

In an embodiment, the computing device described herein may further include, be coupled to, and/or otherwise be associated with one or more computing devices and/or computing systems described below and illustrated in the example embodiments depicted in FIGS. 1-6. For example, in at least one embodiment, the computing device described herein may include, be coupled to, and/or otherwise be associated with wearable computing device, mobile computing device, and/or server system.

In the above embodiment, a wearable computing device, mobile computing device, and/or server system can individually and/or collectively perform the cardiac monitoring and/or cardiac assessment operations (e.g., determining the cardiac score for the user) described herein in accordance with one or more embodiments of the present disclosure. In this embodiment, based at least in part on (e.g., in response to) performing such cardiac assessment operations, the wearable computing device, mobile computing device, and/or server system can further perform, individually and/or collectively, one or more operations described herein that can facilitate alteration (e.g., improvement) of a user's health quality in accordance with one or more embodiments of the present disclosure.

Further, a user may be provided with privacy-related controls allowing the user to make an election as to both if and when systems, programs, or features described herein may enable collection of health-related data and/or user information (e.g., information about a user's social network, social actions, or activities, profession, a user's preferences, or a user's current location), and if the user is sent content or communications that may be of a sensitive or private nature from a server. In addition, certain data may be treated in one or more ways before it is stored or used, so that personally identifiable information is removed. For example, a user's identity may be treated so that no personally identifiable information can be determined for the user, or a user's geographic location may be generalized where location information is obtained (such as to a city, ZIP code, or state level), so that a particular location of a user cannot be determined. Thus, the user may have control over what information is collected about the user, how that information is used, and what information is provided to the user. To that end, any information collected as described herein relating to the user (e.g., personal medical data, health conditions, etc.) is capable of being kept private and confidential and not be improperly used or published.

Moreover, one or more security measures can be implemented to ensure that the demographic data and the physiological data of the user is safeguarded. For example, passcode or fingerprint authentication may be used to control access to the demographic data and the physiological or otherwise personal data of the user. Further, such data of the user can be stored in a privacy enhancing manner and not shared without the express consent of the user. For example, such data can be encrypted to secure the data from unauthorized access.

Example aspects of the present disclosure provide several technical effects, benefits, and/or improvements in computing technology.

### Example Devices and Systems

Referring now to the drawings, FIGS. 1-3 illustrate perspective views of an example wearable computing device 100 according to one or more example embodiments of the present disclosure. In example embodiments, the wearable computing device 100 may include, for example, a wearable physiological monitoring device that can be worn by a user 10 and/or capture one or more types of physiological data of the user (e.g., HR data, motion data (e.g., accelerometer data), body temperature data, respiration rate data, blood pressure data, blood oxygenation level data, electrodermal activity (EDA) data, stress related data).

Furthermore, in an embodiment, the wearable computing device 100 may include a display 102, an attachment component 104, a securement component 106, and a button 108 that can be located on a side of the wearable computing device 100. In an embodiment, two sides of the display 102 can be coupled (e.g., mechanically, operatively) to the attachment component 104. In some embodiments, the securement component 106 can be located on, coupled to (e.g., mechanically, operatively), and/or integrated with the attachment component 104. In these or other embodiments, the securement component 106 can be positioned opposite the display 102 on an opposing end of the attachment component 104. In some embodiments, the button 108 can be located on a side of the wearable computing device 100, underneath the display 102.

Furthermore, the display 102 may include any type of electronic display or screen known in the art. For example, in some embodiments, the display 102 may include a liquid crystal display (LCD) or organic light emitting diode (OLED) display such as, for instance, a transmissive LCD display or a transmissive OLED display. Further, the display 102 can be configured to provide brightness, contrast, and/or color saturation features according to display settings that can be maintained by control circuitry and/or other internal components and/or circuitry of the wearable computing device 100. In some embodiments, the display 102 may include a touchscreen such as, for instance, a capacitive touchscreen. For example, in these embodiments, the display 102 may include a surface capacitive touchscreen or a projective capacitive touch screen that can be configured to respond to contact with electrical charge-holding members or tools, such as a human finger. While the wearable computing device 100 is shown in example embodiments of the present disclosure to have the display 102, it should be understood that, in some embodiments, the wearable computing device 100 does not have any type of display unit.

In some embodiments, the display 102 can be configured to provide (e.g., render) a variety of information such as, for example, the time, the date, physiological data of a user wearing the wearable computing device 100, readings based upon user input, and/or other information. In an embodiment, the physiological data can include, but are not limited to, HR data (e.g., heart beats per minute), motion data (e.g., movement data, accelerometer data), blood pressure data, body temperature data, respiration rate data, blood oxygenation level data, EDA data, stress related data and/or any other physiological data that one of ordinary skill in the art would understand that can be measured by the wearable computing device 100. In some embodiments, the readings based upon user input can include, but are not limited to, activities performed by the user, a sleep schedule of the user, and/or any other metric that one of ordinary skill in the art would understand that can be input by a user into the wearable computing device 100.

The attachment component 104 can be used to attach (e.g., affix, fasten) the wearable computing device 100 to a user thereof (e.g., to the user's 10 body or clothing). In some embodiments, the attachment component 104 can take the form of, for example, a strap, an elastic band, a rope, and/or any other form of attachment one of ordinary skill in the art would understand can be used to attach the wearable computing device 100 to a user. For example, the wearable computing device 100 can be configured as a wrist bracelet, watch, ring, electrode, finger-clip, toe-clip, chest-strap, ankle strap, and/or a device placed in a pocket. In additional or alternative embodiments, the wearable computing device 100 can be embedded in something in contact with the user 10 such as, for instance, clothing, a mat that can be positioned under the user 10, a blanket, a pillow, and/or another accessory.

The securement component 106 can facilitate attachment of attachment component 104 upon a user of wearable computing device 100. In some embodiments, the securement component 106 can include, but is not limited to, a pin and hole locking mechanism (e.g., a buckle), a magnet system, a lock, a clip, and/or any other type of securement that one of ordinary skill would understand can be used to facilitate attachment of the wearable computing device 100 to a user. In an embodiment, the wearable computing device 100 does not include the securement component 106. For example, in an embodiment, the wearable computing device 100 can be secured to a user with a strap that can be tied around the user's wrist and/or another suitable appendage.

The button 108 can allow for a user to interact with the wearable computing device 100 and/or allow for the user to provide a form of input into wearable computing device 100. For instance, as described above, in example embodiments, the wearable computing device 100 can include a screen such as, for example, a touch screen that can receive inputs through (e.g., by way of) the touch of the user. In additional or alternative embodiments, the wearable computing device 100 can include a microphone that can receive inputs through (e.g., by way of) voice commands of a user.

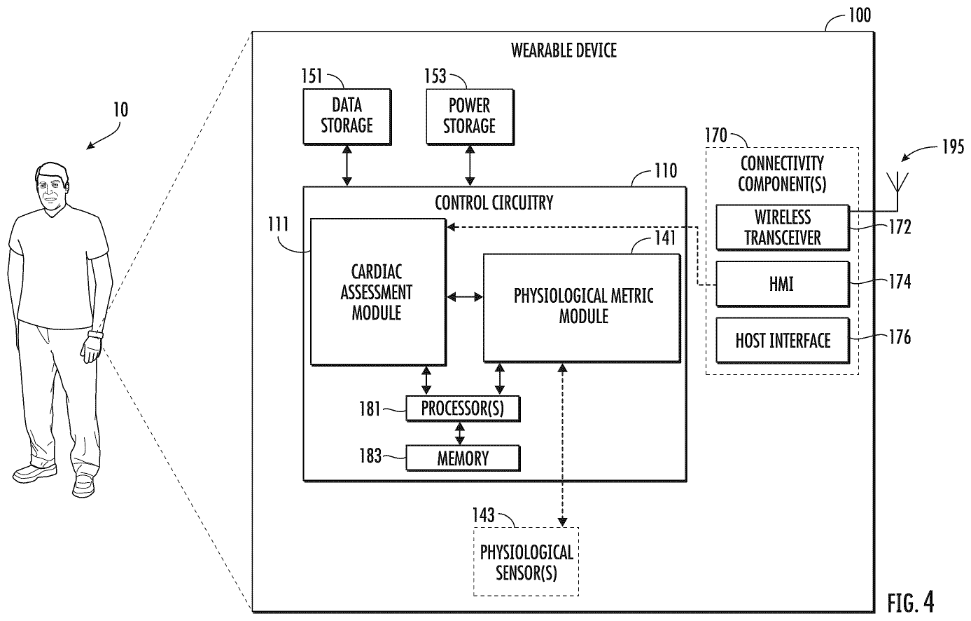

Referring now to FIG. 4, a block diagram of the wearable computing device 100 according to one or more example embodiments of the present disclosure is illustrated. That is, for instance, FIG. 4 illustrates a block diagram of one or more internal and/or external components of the wearable computing device 100 according to one or more example embodiments of the present disclosure.

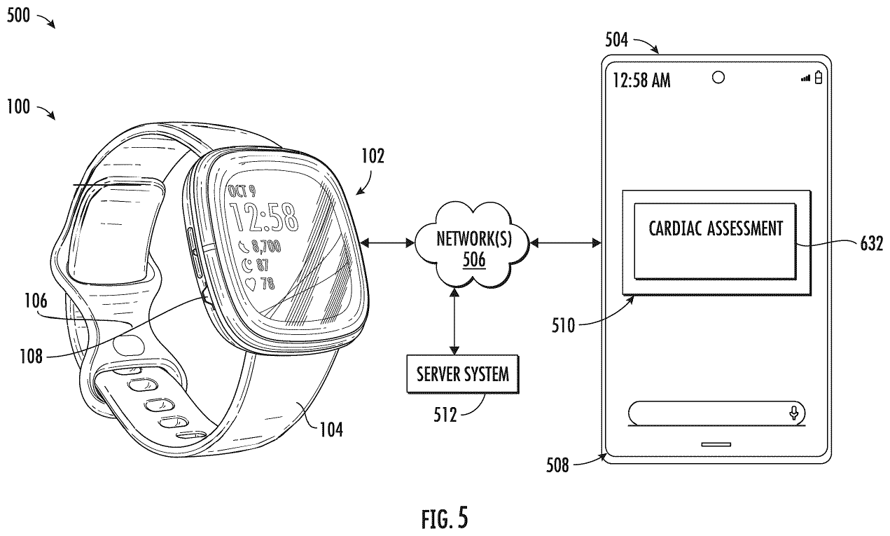

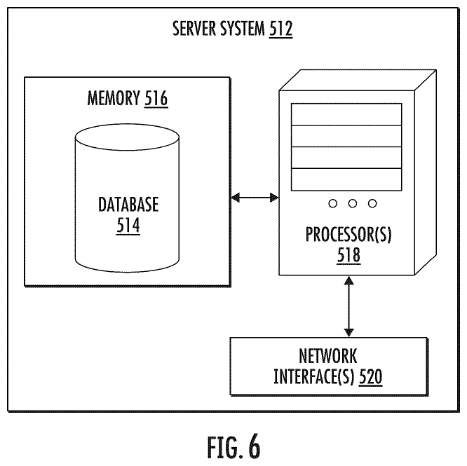

Although certain embodiments are disclosed herein in the context of wearable physiological monitoring devices, it should be appreciated that the present disclosure is not so limiting. For example, it should be understood that the physiological monitoring and the cardiac assessment principles and features disclosed herein can be performed and/or implemented using any suitable type of computing device or combination of computing devices such as, for example, a client computing device, a laptop, a tablet, a server (e.g., a server system 512 described below and depicted in FIG. 6), the wearable computing device 100, a mobile computing device 504, such as a smartphone (e.g., as described below and depicted in FIG. 5), and/or another computing device, whether wearable or not.

As shown in FIG. 4, the wearable computing device 100 may include a wearable physiological monitoring device that can be worn by a user 10 and/or can be configured to gather data regarding activities performed by user 10 and/or a physiological state of the user. In some embodiments, the data may include motion data regarding user's 10 movements and/or physiological data obtained by measuring various physiological characteristics of the user 10 (e.g., heart rate, respiratory data, body temperature, blood oxygen levels, perspiration levels, movement data).

The wearable computing device 100 can include control circuitry 110. Although certain modules and/or components are illustrated as part of the control circuitry 110 in the diagram of FIG. 4, it should be understood that the control circuitry 110 associated with the wearable computing device 100 and/or other components or devices in accordance with example embodiments of the present disclosure can include additional components and/or circuitry such as, for instance, one or more additional components of the illustrated components depicted in FIG. 4. Furthermore, in certain embodiments, one or more of the illustrated components of the control circuitry 110 can be omitted and/or different than that shown in FIG. 4 and described in association therewith.

The term “control circuitry” is used herein according to its broad and/ordinary meaning and can include any combination of software and/or hardware elements, devices, and/or features that can be implemented in connection with operation of the wearable computing device 100. Furthermore, the term “control circuitry” can be used substantially interchangeably in certain contexts herein with one or more of the terms “controller,” “integrated circuit,” “IC,” “application-specific integrated circuit,” “ASIC,” “controller chip,” or the like.

The control circuitry 110 may include one or more processors 181, data storage devices, and/or electrical connections. In an embodiment, the control circuitry 110 can be implemented on a system on a chip (SoC), however, those skilled in the art will recognize that other hardware and/or firmware implementations are possible.

In one or more embodiments, the processor(s) 181 can be configured to execute computer-readable instructions that, when executed, cause the wearable computing device 100 to perform one or more operations. In at least an embodiment, the processor(s) 181 can be configured to execute operational code (e.g., instructions, processing threads, software) for the wearable computing device 100 such as, for instance, firmware or the like. In the example embodiment depicted in FIG. 4, the processor(s) 181 can each be a central processing unit (CPU), microprocessor, microcontroller, integrated circuit (e.g., an application-specific integrated circuit (ASIC)), and/or another type of processing device. In this or another example embodiment, the processor(s) 181 can be coupled to (e.g., electrically, communicatively, physically, operatively) to one or more components of the control circuitry 110 and/or the wearable computing device 100 such that the processor(s) 181 can facilitate one or more operations in accordance with the embodiments described herein.

In an embodiment, as shown in FIG. 4, the computer-readable instructions and/or operational code that can be executed by the processor(s) 181 can be stored in one or more data storage devices of the wearable computing device 100, such as a memory 183 of the wearable computing device 100. In some embodiments, the memory 183 can be coupled to (e.g., electrically, communicatively, physically, operatively) to one or more components of the control circuitry 110 and/or the wearable computing device 100 such that the memory 183 can facilitate one or more operations in accordance with the embodiments described herein.

The memory 183 can store computer-readable and/or computer executable entities (e.g., data, information, applications, models, algorithms) that can be created, modified, accessed, read, retrieved, and/or executed by each of the processor(s) 181. In some embodiments, the memory 183 can constitute, include, be coupled to (e.g., operatively), and/or otherwise be associated with a computing system and/or media such as, for example, one or more computer-readable media, volatile memory, non-volatile memory, random-access memory (RAM), read only memory (ROM), hard drives, flash drives, and/or other memory devices. In these or other embodiments, such one or more computer-readable media can include, constitute, be coupled to (e.g., operatively), and/or otherwise be associated with one or more non-transitory computer-readable media.

The control circuitry 110 may include a cardiac assessment module 111, a physiological metric module 141, and/or other modules and/or data that can be used to facilitate one or more operations described herein. The cardiac assessment module 111 may include one or more hardware and/or software components and/or features that can be configured to perform a cardiac assessment of the user 10, as described further below. In some embodiments, to perform such assessment(s), the cardiac assessment module 111 can use inputs from the physiological metric module 141, as described further below.

In an embodiment, the wearable computing device 100 can include one or more physiological sensors 143 that can be configured to collect the physiological data of the user in accordance with various embodiments disclosed herein. For example, the physiological sensors 143 may include a heart rate sensor, photoplethysmography (PPG) sensor, and/or other physiological sensors. In some embodiments, the physiological sensor(s) 143 can be disposed on, coupled to, embedded and/or integrated in, and/or otherwise be associated with the wearable computing device 100 such that the physiological sensor(s) 143 can be in contact with or substantially in contact with human skin when the wearable computing device 100 is worn by a user. For example, in embodiments the physiological sensor(s) can be disposed on, coupled to, and/or otherwise be associated with an interior or skin-side of the wearable computing device 100 (e.g., a side of the wearable computing device 100 that contacts, touches, and/or faces the skin of the user). In additional and/or alternative embodiments, the wearable computing device 100 can be configured to receive the physiological data of the user from one or more physiological sensors 143 external to (i.e., not embedded and/or integrated in) the wearable computing device 100.

In an embodiment, the physiological metric module 141 can, for example, be communicatively coupled with the physiological sensor(s) 143 such that the physiological metric module 141 can receive the physiological data of the user 10 collected by the physiological sensor(s) 143. The physiological metric module 141 can, for example, calculate physiological metrics, including but not limited to, the cardiac metric(s), of the user 10 based on the physiological data of the user 10 (e.g., according to known physiological metric calculations).

In some embodiments, the wearable computing device 100 can be configured to analyze and/or interpret the collected physiological data to perform a cardiac assessment of the user 10 of the wearable computing device 100, as described further below. In additional and/or alternative embodiments, the wearable computing device 100 can be configured to communicate with another computing device or server that can perform the cardiac assessment of the user 10 of the wearable computing device 100 according to embodiments described herein.

In the example embodiment depicted in FIG. 4, the wearable computing device 100 can include one or more data storage components 151. The data storage component(s) 151 may include any suitable or desirable type of data storage such as, for instance, solid-state memory, which can be volatile or non-volatile. In some embodiments, such solid-state memory of the wearable computing device 100 may include any of a wide variety of technologies such as, for instance, flash integrated circuits, phase change (PC) memory, phase change (PC) random-access memory (RAM), programmable metallization cell RAM (PMC-RAM or PMCm), ovonic unified memory (OUM), resistance RAM (RRAM), NAND memory, NOR memory, EEPROM, ferroelectric memory (FeRAM), MRAM, or other discrete NVM (non-volatile solid-state memory) chips. In some embodiments, the data storage component(s) 151 can be used to store system data, such as operating system data and/or system configurations or parameters. In some embodiments, the wearable computing device 100 can include data storage utilized as a buffer and/or cache memory for operational use by the control circuitry 110.

The data storage component(s) 151 can include various sub-modules that can be implemented to facilitate the physiological monitoring and the cardiac assessment principles and features disclosed herein. For example, in at least an embodiment, the data storage component(s) 151 can include one or more sub-modules that can include, but not limited to: an information collection module (e.g., the physiological metric module 141) that can manage the collection of the physiological data, demographic data, and/or anthropometric data relevant to the cardiac assessment; a heart rate determination module that can determine values and/or patterns of one or more types of heart rates of the user 10; the cardiac assessment module 111; a sleep detection module that can detect an attempt or onset of sleep by the user 10; a presentation module that can manage presentation of information to the user 10 that can be associated with the cardiac assessment; a feedback management module for collecting and interpreting any input data and/or feedback received from the user 10; and/or another sub-module.

The wearable computing device 100 can further include a power storage module 153 (denoted in FIG. 4 as “power storage 153”), which may include a rechargeable battery, one or more capacitors, or other charge-holding device(s). In some embodiments, the power stored by the power storage module 153 can be utilized by the control circuitry 110 for operation of the wearable computing device 100, such as for powering the display 102. In some embodiments, the power storage module 153 can receive power over a host interface of the wearable computing device 100 (e.g., via one or more host interface circuitry and/or components 176 (denoted as “host interface 176” in FIG. 4)) and/or through other means.

The wearable computing device 100 can further include one or more connectivity components 170, which can include, for example, a wireless transceiver 172. The wireless transceiver 172 can be communicatively coupled to one or more antenna devices 195, which can be configured to wirelessly transmit and/or receive data and/or power signals to and/or from the wearable computing device 100 using, but not limited to, peer-to-peer, WLAN, and/or cellular communications. For example, the wireless transceiver 172 can be utilized to communicate data and/or power between the wearable computing device 100 and an external computing device, which can be configured to interface with the wearable computing device 100. In certain embodiments, the host interface 176 can include, for example, wired and/or wireless interface components that can communicatively couple the wearable computing device 100 with the external computing device to receive data and/or power therefrom and/or transmit data thereto. The host interface circuitry and/or component(s) 176 according to example embodiments can utilize and/or otherwise be associated with any suitable or desirable communication protocol and/or physical connector such as, for instance, universal serial bus (USB), micro-USB, Wi-Fi, Bluetooth, Fire Wire, PCIe, or the like. For wireless connections, the host interface 176 according to example embodiments can be incorporated with the wireless transceiver 172.

The connectivity component(s) 170 can further include one or more HMIs 174 that can be used by the wearable computing device 100 to receive input data from user 10 and/or provide output data to the user 10. For instance, in some embodiments, the HMI 174 of the wearable computing device 100 may include a touchscreen display that can be configured to provide (e.g., render) output data to the user 10 and/or to receive user input through user contact with the touchscreen display. In some embodiments, the HMI(s) 174 can further constitute and/or include one or more buttons or other input/output components or features.

Referring now to FIG. 5, a diagram of an example cardiac assessment system 500 according to one or more example embodiments of the present disclosure is illustrated. As shown, the cardiac assessment system 500 depicted in FIG. 5 illustrates an example networked relationship between the wearable computing device 100 and a mobile computing device 504 in accordance with one or more embodiments.

With reference to the example embodiment described above and depicted in FIG. 4, the wearable computing device 100 according to example embodiments of the present disclosure can perform a cardiac assessment of the user 10 and/or perform operation(s) to facilitate alteration (e.g., improvement) of the user's 10 cardiac health based on the cardiac assessment. As such, in certain embodiments the wearable computing device 100 can be capable of and/or configured to collect physiological sensor data of the user 10 and/or perform the cardiac assessment and/or operation(s) using such readings.

However, in additional and/or alternative embodiments, the wearable computing device 100 and/or another electronic and/or computing device that can be used to detect the physiological data of the user 10, can be in communication with the mobile computing device 504. In these and/or other embodiments, the mobile computing device 504 can be configured to use the physiological data of the user 10 to perform the cardiac assessment of the user 10 according to one or more embodiments described herein. In these and/or other embodiments, based at least in part on (e.g., in response to) performing the cardiac assessment, the mobile computing device 504 can perform one or more operations described herein to facilitate alteration (e.g., improvement) of the user's 10 cardiac health.

The wearable computing device 100 can also be configured to collect the physiological data of the user 10 using embedded sensors and/or external devices, as described throughout the present disclosure, and communicate or relay such data over one or more networks 506 to other devices. This includes, in some embodiments, relaying data to devices capable of serving as Internet-accessible data sources, thus permitting the collected data to be viewed, for example, using a web browser or network-based application at, for instance, the mobile computing device 504. For example, the wearable computing device 100, e.g., while being worn by the user 10, can capture, calculate, and/or store the physiological data of the user 10 using the physiological sensor(s) 143. The wearable computing device 100 can then transmit (e.g., periodically or continuously) data representative of the physiological data over the network(s) 506 to the mobile computing device 504 and/or a server system 512 where the data can be stored, processed, and visualized by the user 10 and/or another entity (e.g., a health care professional). Accordingly, in an embodiment, the mobile computing device 504 may be configured to generate an intelligent notification 510 and provide the intelligent notification 510 to the user 10, e.g., via the display 508 or a second computing device. In some embodiments, the intelligent notification 510 can include the cardiac assessment, which will be described further below.

In one or more embodiments, the communication between the wearable computing device 100 and the mobile computing device 504 can be facilitated by the network(s) 506. In some embodiments, the network(s) 506 may include, for instance, one or more of an ad hoc network, a peer-to-peer communication link, an intranet, an extranet, a virtual private network (VPN), a local area network (LAN), a wireless LAN (WLAN), a wide area network (WAN), a wireless WAN (WWAN), a metropolitan area network (MAN), a portion of the Internet, a portion of the public switched telephone network (PSTN), a cellular telephone network, and/or any other type of network. In some embodiments, the communication between the wearable computing device 100 and the mobile computing device 504 can also be performed through a direct wired connection. In these or other embodiments, this direct-wired connection can be associated with any suitable or desirable communication protocol and/or physical connector such as, for instance, universal serial bus (USB), micro-USB, Wi-Fi, Bluetooth, Fire Wire, PCIe, or the like.

In example embodiments, a variety of computing devices can be in communication with the wearable computing device 100 to facilitate the user's cardiac assessment and/or alteration (e.g., improvement). Although the mobile computing device 504 is depicted as a smartphone in the example embodiment illustrated in FIG. 5, it should be understood that the present disclosure is not so limiting. For instance, the mobile computing device 504 according to example embodiments may include, for example, a smartphone with a display 508 as depicted in FIG. 5, a personal digital assistant (PDA), a mobile phone, a tablet, a personal computer, a laptop computer, a smart television, a video game console, and/or another computing device that can be external to the wearable computing device 100.

Referring particularly to FIGS. 5 and 6, the wearable computing device 100 can transmit the physiological data of the user 10 to the server system 512 (e.g., via the network(s) 506). In this embodiment, the server system 512 can analyze the received physiological data to perform the cardiac assessment and/or can use the received physiological data to update a user profile for the user 10 that can be stored in a database 514 (e.g., a log) of a memory 516 of the server system 512.

In some embodiments, the server system 512 can be implemented on one or more standalone data processing apparatuses or a distributed network of computers. In some embodiments, the server system 512 can employ various virtual devices and/or services of third-party service providers (e.g., third-party cloud service providers) to provide the underlying computing resources and/or infrastructure resources of the server system 512. In some embodiments, the server system 512 can include, but is not limited to, a handheld computer, a tablet computer, a laptop computer, a desktop computer, or a combination of any two or more of these data processing devices or other data processing devices.

The server system 512 can include one or more processors 518 such as, for instance, one or more CPUs. In these or other embodiments, the server system 512 can include one or more network interfaces 520 that can include, for example, an input/output (I/O) interface to the mobile computing device 504 and/or the wearable computing device 100. In some embodiments, the server system 512 can include one or more communication buses for interconnecting these components.

The memory 516 according to example embodiments can include high-speed random-access memory such as, for instance, DRAM, SRAM, DDR RAM, or other random-access solid-state memory devices; and, optionally, can include non-volatile memory such as, for example, one or more magnetic disk storage devices, one or more optical disk storage devices, one or more flash memory devices, or one or more other non-volatile solid state storage devices. The memory 516, optionally, can include one or more storage devices that can be remotely located from the processor(s) 518 (e.g., processing unit(s)). Further, the memory 516, or alternatively the non-volatile memory within the memory 516, can include a non-transitory computer readable storage medium. In some embodiments, the memory 516, or the non-transitory computer readable storage medium of the memory 516, can store one or more programs, modules, and data structures. In these embodiments, such programs, modules, and data structures can include, but not be limited to, one or more of an operating system that can include procedures for handling various basic system services and for performing hardware dependent tasks.

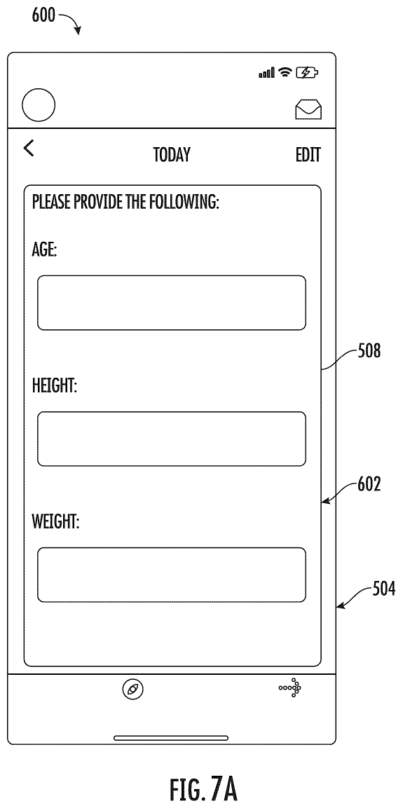

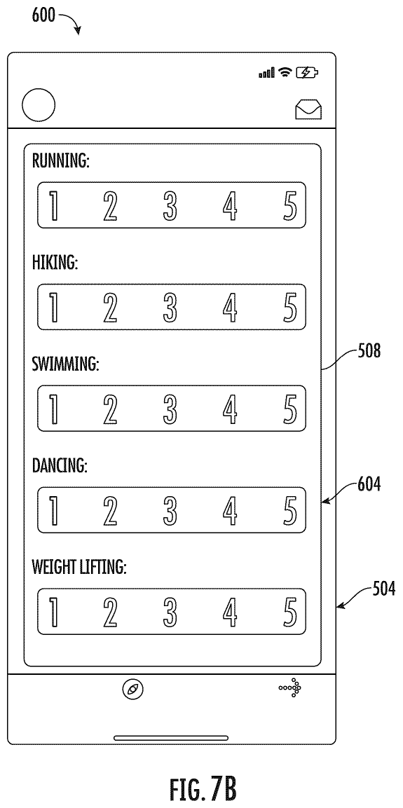

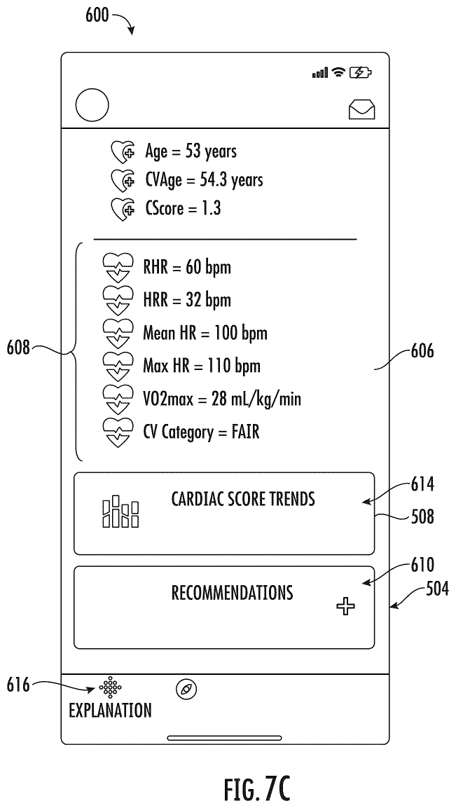

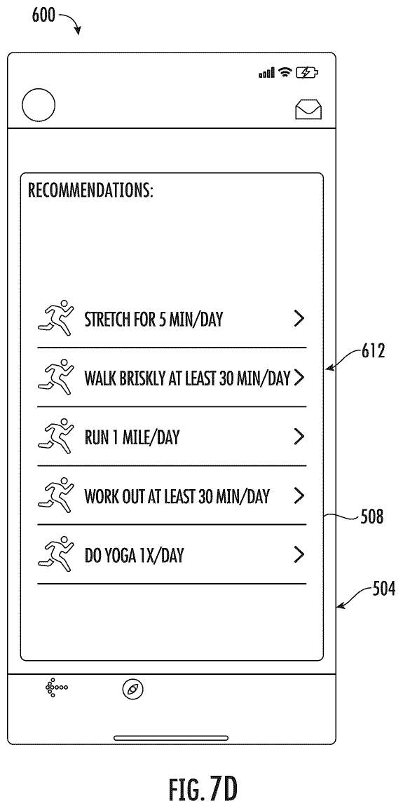

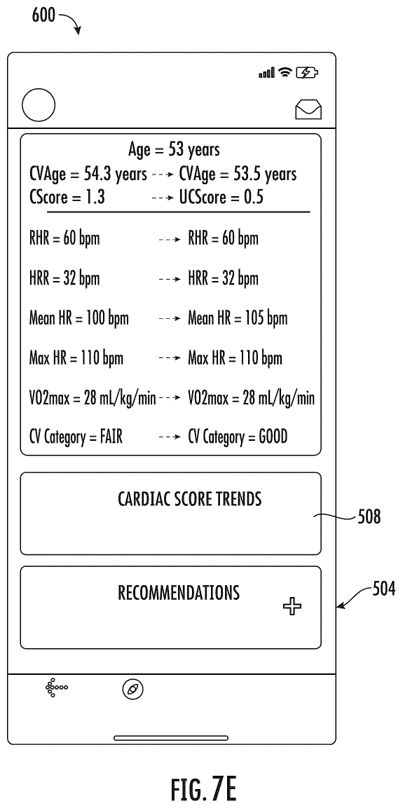

Referring now to FIGS. 7A-7E, various views of a cardiac health program 600 being implemented on the wearable computing device 100, the mobile computing device 504, and/or the server system 512 as described herein are illustrated. In particular, the cardiac health program 600 may be implemented on a computer application programmed in any of the wearable computing device 100, the mobile computing device 504, and/or the server system 512. Accordingly, in an embodiment, the cardiac health program 600 is configured to provide users with proactive cardiac health assessment and monitoring. In addition, the cardiac health program 600 may educate users about their predicted cardiac health, provide an actionable assessment about their predicted cardiac health, and allow users to easily understand the magnitude and importance that adjusting lifestyle choices can have on their cardiac health.

Referring particularly to FIG. 7A, the cardiac health program 600 may obtain demographic data from the user 10. The demographic data may be obtained via an HMI (e.g., a touchscreen display, a keyboard component, etc.) configured to detect a user input specifying the demographic data. In an embodiment, the display 508 of the mobile computing device 504 may, for example, display a request screen 602 prompting the user 10 to enter the demographic data and including respective input boxes configured to receive inputs specifying the corresponding demographic data. In additional or alternative embodiments, the display 102 of the wearable computing device 100 may display the request screen 602 and prompt the user 10 to enter the demographic data. The demographic data may include an age (i.e., an actual or chronological age) of the user 10. The demographic data may further include any other suitable data (e.g., sex, gender, race, etc.) associated with user cardiac health.

In additional or alternative embodiments, the cardiac health program 600 may obtain anthropometric data from the user 10. The anthropometric data may be obtained via the HMI. For example, as shown, the request screen 602 may further specify the anthropometric data and include respective input boxes configured to receive inputs specifying the corresponding anthropometric data, as discussed above. The anthropometric data may include, for example, height, weight, body mass index (BMI), and/or any other suitable data associated with user cardiac health.

Moreover, the cardiac health program 600 may obtain physiological data of the user 10. For example, the physiological data may be obtained via the physiological sensor(s) 143 of the wearable computing device 100. The physiological data includes one or more cardiac metrics of the user 10. The cardiac metric(s) can include, but are not limited to, a resting heart rate (RHR), an average heart rate (MeanHR), a maximum heart rate (MaxHR), a heart rate recovery (HRR), and a maximal oxygen consumption (VO2max). The cardiac metric(s) may be data obtained by the one or more physiological sensors 143 and/or derived from the data obtained via the one or more physiological sensors 143 (e.g., calculated via the physiological metric module 141, as discussed above).

Further, in embodiments, the cardiac health program 600 may obtain activity (e.g., exercise) data for the user 10. For example, the request screen 602 may further include input boxes (not shown) configured to receive inputs specifying the activity data. The activity data can include, but is not limited to, as type of activity (such as exercises), a duration of the activity, an intensity of the activity (e.g., determined based on heart rate data obtained while the user 10 performs the activity), a location of the activity, a frequency of reoccurrence of the activity (e.g., once per day, once per week, etc.), and any other suitable data associated with the activity and/or the physiological data of the user 10 while performing the activity. As another example, at least some of the activity data may be derived from the physiological data of the user 10 (e.g., based on HR data, motion data, etc.). The cardiac health program 600 may then monitor (e.g., track) the activity data for the user 10 over a period of time (e.g., 1 day, 1 week, 1 month, 3 months, 6 months, 1 year, etc.) so as to have a more accurate representation of the user's activity data characteristics and tendencies. That is, the cardiac health program 600 may maintain historical activity data for the user 10.

In addition, the cardiac health program 600 may obtain activity preferences for the user 10. In some embodiments, the display 508 of the mobile computing device 504 may, for example, display a preferences screen 604 providing one or more questions for the user 10 to answer relating to activity preferences, as shown in FIG. 7B. For example, as shown, the user may be prompted to rate (e.g., on a scale of 1 to 5, with 1 being strongly dislike and 5 being strongly like) certain activities (such as running, hiking, dancing, swimming, weight lifting, etc.). The cardiac health program 600 may use this subjective data to improve recommendations provided to the user 10, as described below.

Referring particularly to FIG. 7C, the cardiac health program 600 is configured to determine a cardiac score (CScore) of the user 10 and to provide the cardiac score (CScore) to the user 10. For example, as shown, the display 508 of the mobile computing device 504 may display a cardiac health screen 606 specifying the cardiac score (CScore). In additional and/or alternative embodiments, the display 102 of the wearable computing device 100 may display the cardiac health screen 606. Displaying the cardiac score (CScore) to the user 10 allows the user to easily view and track the cardiac score (CScore) and the cardiac metric(s) 608 associated with the cardiac score (CScore). Accordingly, the user 10, by viewing the display 508, can be made easily aware of the cardiac metric(s) 608 associated with the current cardiac score (CScore). The cardiac score (CScore) is configured to assess cardiac health of the user. For example, a positive value for the cardiac score (CScore) may represent superior cardiac health for a particular age, and a negative value for the cardiac score (CScore) may indicate inferior cardiac health for the particular age.

Further, in embodiments, as shown, the cardiac health program 600 may be configured to categorize the cardiac score (CScore) for the user 10. In embodiments, the cardiac health program 600 can, for example, categorize the cardiac score (CScore) based, at least in part, on the demographic data of the user 10 (e.g., according to known percentile score and/or bucketing techniques). For example, categories (CV Category) may be defined by percentile ranges of cardiac score (CScore) for users within respective age groups (e.g., defined by successive age ranges (e.g., a 5-year range)). In additional and/or alternative embodiments, the cardiac health program 600 can categorize the cardiac score (CScore) based on the anthropometric data. For example, the categories (CV Category) may be defined by percentile ranges of cardiac score for users within respective age groups and/or having BMI within respective ranges.

In certain embodiments, the categories may be identified by text strings, such as “Poor”, “Fair”, “Average”, “Good”, “Very Good”, and “Excellent”. In alternative embodiments, the categories (CV Category) may be identified on a scale (e.g., of 1 to 6, with 1 being poor and 6 being excellent). As such the categories (CV Category) may be configured to indicate cardiac health of the user 10 relative to cardiac health of all users 10. In some embodiments, the display 508 of the mobile computing device 504 may display the category (CV Category) of the cardiac score (CScore), as shown in FIG. 7C, so as to assist users 10 in understanding their cardiac health relative to other users.

Furthermore, the cardiac health program 600 may be configured to provide historical cardiac scores for the user 10. For example, in response to the user 10 may selecting a “TRENDS” button 614 on the display 508, the display 508 may display cardiac scores (CScores) for a period of time (e.g., 1 month, 6 months, 1 year, 3 years, etc.). Displaying the historical cardiac scores allows the user 10 to easily understand the change in their cardiac score (CScores) in response to changes in their activities. For example, the historical cardiac scores may further specify a change in activity associated with the corresponding historical cardiac score. As such, the cardiac health program 600 may be further configured to educate users about the influence of various activities over a period of time on their cardiac health.

Moreover, the cardiac health program 600 may be configured to determine recommendations regarding exercise for the user. For example, in response to the user 10 selecting a “RECOMMENDATIONS” button 610 on the display 508, the display 508 may display a recommendation screen 612 including one or more recommendations, as shown in FIG. 7D. The recommendations may be manually selected by the user 10 and may include a measurable amount (e.g., how often and how much), relevancy (e.g., likelihood to improve one or more cardiac metrics), and/or a defined time for a recommended activity. The recommendations may be determined based on one or more machine-learned models, a look-up table, or the like, that associates various activities with various changes in the cardiac metric(s) 608.

For example, in an embodiment, the cardiac health program 600 may include one or more machine-learned models that can include at least one recommendation generation model. In such embodiments, the recommendation generation model(s) is configured to generate one or more recommendations regarding exercise for the user using machine learning. For example, in an embodiment, the machine-learned model(s) may be trained to output the recommendation(s) in response to receiving the cardiac score (CScore), the physiological data of the user 10, the historical activity data for the user 10, and/or the activity preferences of the user 10. In such embodiments, the recommendation(s) can include natural language recommendations of one or more exercises that can be performed by the user to reach and/or remain within a specified category (CV Category).

The machine-learned model(s) as described herein may include neural networks (e.g., deep neural networks) or a generative model (e.g., large language models (LLM), non-linear models or linear models, decision tree based models, support vector machines, hidden Markov models, Bayesian networks, and/or k-means clustering models, etc.). Example machine-learned models can also use other architectures in lieu of or in addition to those models specifically mentioned herein.

As such, the recommendations may be configured to update the cardiac metric(s) 608 for the user 10 so as to influence the cardiac score (CScore) of the user 10 to reach and/or remain within a specified category (CV Category), such as “Very Good”.

That is, in some circumstances the cardiac health program 600 may recommend that the user 10 perform new activities so as to improve their cardiac score (CScore), while in other circumstances, the cardiac health program 600 may recommend that the user 10 continue performing current activities so as to maintain their cardiac score (CScore). In some embodiments, the cardiac health program 600 may be configured to select activities that are preferred and/or previously completed by the user 10 so as to increase a likelihood of the user 10 performing the recommended activity and thereby increasing a likelihood of the user 10 improving or maintaining their cardiac score (CScore).

In addition, in some embodiments, the cardiac health program 600 may be configured to prompt the user 10 to perform an activity. In an embodiment, the display 508 may display a prompt identifying the activity to the user 10. In such an embodiment, the prompt may be manually selected by the user 10 (e.g., via the HMI 174) when the user 10 is able to perform the activity. The prompt may, for example, specify one or more recommendations selected by the user 10. As another example, the prompt may specify one or more activities maintained in a look-up table, or the like, that are associated with various cardiac metrics 608. That is, the activities maintained in the look-up table may be specified so as to cause changes in one or more specified cardiac metrics 608. In some embodiments, the prompt may be automatically displayed to the user 10 in response to a trigger (e.g., a specified time of day (e.g., specified via the activity preferences), after a period of time (e.g., during which the user 10 is stationary), a change in the cardiac score (CScore), a change in activity level, a change in weight, and/or any other suitable trigger).

In response to the user 10 selecting the prompt, the cardiac health program 600 may be configured to obtain updated physiological data for the user 10 (e.g., via the physiological sensors 143, as discussed above) while the user 10 performs the activity. The updated physiological data includes the cardiac metric(s) 608. In some embodiments, the cardiac health program 600 may be configured to determine an updated cardiac score (UCScore) based on the updated physiological data for the user 10. In such embodiments, the display 508 may display a comparison between the cardiac score (CScore) and the updated cardiac score (UCScore), as shown in FIG. 7E. Comparisons between the physiological data for the user 10 and the updated physiological data for the user 10 may also be displayed via the display 102, as shown. Displaying the comparisons to the user 10 can educate the user 10 on how performing various activities influences changes to various cardiac metrics 608 and the cardiac score (CScore).

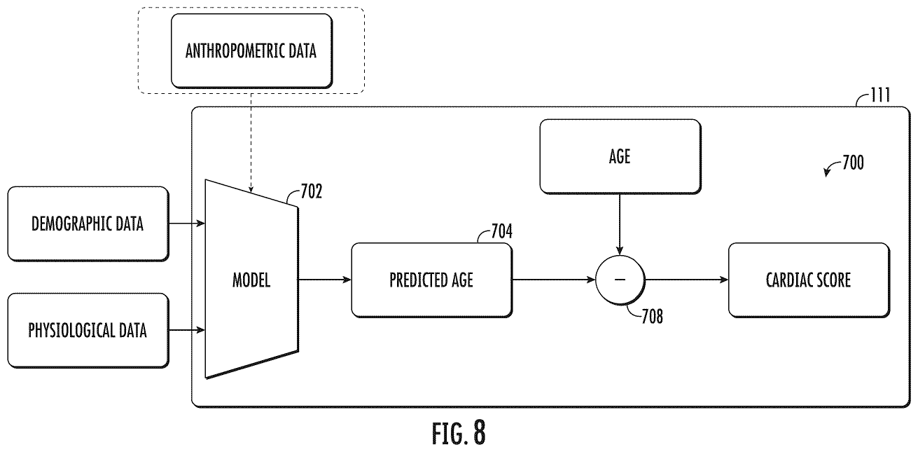

Referring now to FIG. 8, the cardiac assessment module 111 is configured to perform a cardiac scoring operation 700. For example, in an embodiment, the cardiac assessment module 111 inputs the demographic data and the physiological data, and more particularly, the cardiac metric(s) 608, of the user 10 to a model 702. In certain embodiments, the cardiac assessment module 111 may be configured to also input the anthropometric data to the model 702 and/or any other suitable health data of the user 10, such as sleep data. In such an example, the cardiac assessment module 111 can receive a predicted cardiac age 704 as a prediction output by the model 702. That is, the model 702 can be trained to accept the demographic data, the physiological data, and/or the anthropometric data as inputs and to generate an output of a predicted cardiac age 704 for the user 10 based on such inputs. The cardiac assessment module 111 is further configured to determine the cardiac score (CScore) based on a difference 708 between the predicted cardiac age 704 and the age (e.g., specified via the demographic data, as discussed above) of the user 10.

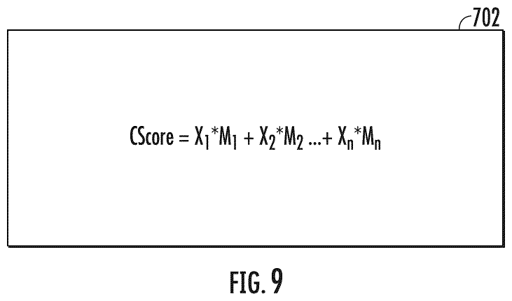

In an embodiment, the model 702 can be a software program loaded in memory and executed by a processor included in a computing device (e.g., the wearable computing device 100, the mobile computing device 504 and/or the server system 512). In some embodiments, the model 702 is a statistical model, such as a linear regression model, as shown in FIG. 9. In such embodiments, the model 702 may be trained with training data included in a biomedical database (e.g., maintained on the server system 512). The training data may include demographic, physiological, and/or anthropometric data of various users. The linear regression model 702 can be trained with the training data to determine coefficients (X₁, X₂, . . . Xₙ) that output the predicted cardiac age 704 for specified metrics (M₁, M₂, . . . Mₙ) input to the linear regression model 702. The specified metrics (M₁, M₂, . . . Mₙ) may be determined by applying Least Absolute Shrinkage and Selection Operator (LASSO) regression and/or Variance Inflation Factor (VIF) to demographic, physiological, and/or anthropometric data to exclude metrics (e.g., height, weight, sex, one or more cardiac metrics, etc.) that are highly correlated with each other, so as to improve the model's 702 stability and interpretability. Further, the training data can be pre-processed (e.g., according to known data processing techniques, such as z-scoring) to account for variability across the training data. The training data can include, but is not limited to, physiological data for various users specifying the cardiac metric(s), and demographic data, and more particularly the age, for the various users, and/or anthropometric data for various users.

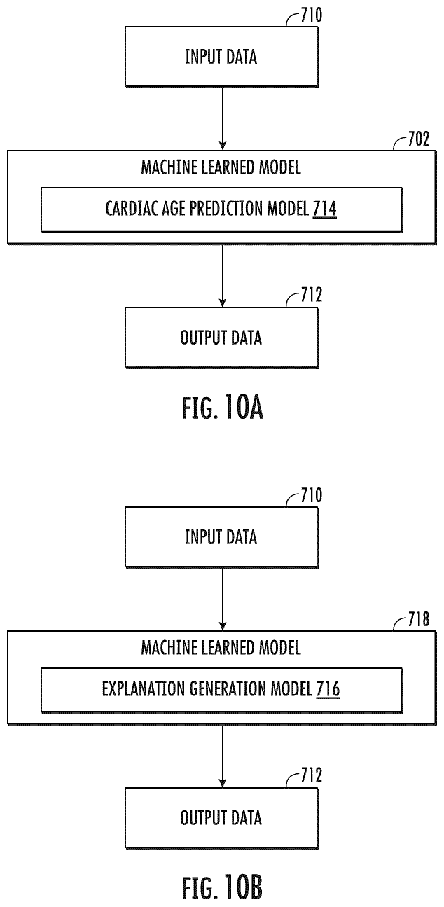

In alternative embodiments, the model 702 may be a machine-learned model, as shown in FIG. 10A. In such embodiments, the machine-learned model 702 can be trained to receive input data 710 and generate output data 712. For example, the model 702 can include a cardiac age prediction model 714 that is operable to predict a cardiac age of a user 10 based on demographic data, physiological data, and/or anthropometric data of the user 10. That is, the model 702 may be further trained to output the predicted cardiac age 704 in response to receiving the demographic data, physiological data, and/or anthropometric data of the user 10 as the input data 710, as shown in FIG. 10A. The machine-learned model 702 may be trained with ground truth data (i.e., data about a real-world condition or state). The ground truth data may include, but is not limited to, the training data discussed above in relation to the linear regression model. Training the machine-learned model 702 can include updating weights and biases via suitable techniques, such as back-propagation.

The machine-learned model 702 can be or include one or multiple machine-learned models or model components. Example machine-learned models can include neural networks (e.g., deep neural networks) or a generative model (e.g., large language models (LLM), non-linear models or linear models, decision tree based models, support vector machines, hidden Markov models, Bayesian networks, and/or k-means clustering models, etc. Example machine-learned models can also use other architectures in lieu of or in addition to those models specifically mentioned herein.

Neural networks, such as those described herein, can include feed-forward neural networks, recurrent neural networks (e.g., long short-term memory recurrent neural networks), convolutional neural networks, and/or other forms of neural networks. Some example machine-learned models can leverage an attention mechanism such as self-attention. For example, some example machine-learned models can include multi-headed self-attention models (e.g., transformer models). In another embodiment, the machine learning models described herein may include a rule-based approach, wherein actions are chosen based on a predetermined set of if-then rules or mathematical expressions with pre-defined parameters.

In additional and/or alternative embodiments, as shown, the cardiac health program 600 may be configured to determine one or more explanations providing context for the cardiac score (CScore) given the physiological data of the user 10. The display 508 may, for example, display the one or more explanations to the user 10 in response to the user selecting an “EXPLANATIONS” button 616 (as shown in FIG. 7C). In such embodiments, an additional machine-learned model 718 can include an explanation generation model 716 that is operable to generate an analysis including one or more explanations of the cardiac score (CScore) and the physiological data. That is, the additional machine-learned model 718 may be trained to output the explanation(s) as the output data 712 in response to receiving the cardiac score (CScore) and the physiological data of the user 10 as the input data 710, as shown in FIG. 10B. For example, the explanations can include a natural language explanation of the cardiac score (CScore) given the physiological data of the user 10. As one example, the explanation may indicate “YOU HAVE A LOWER RESTING HEART RATE THAN OTHER USERS OF YOUR AGE, WHICH IS IMPROVING YOUR CARDIAC SCORE.” Further, the explanations may indicate “TO IMPROVE YOUR CARDIAC SCORE, YOU NEED TO INCREASE YOUR MEAN HEART RATE.” That is, the explanations may provide context to allow the user 10 to interpret the cardiac metric(s) 608 and/or the cardiac score (CScore).

### Example Methods

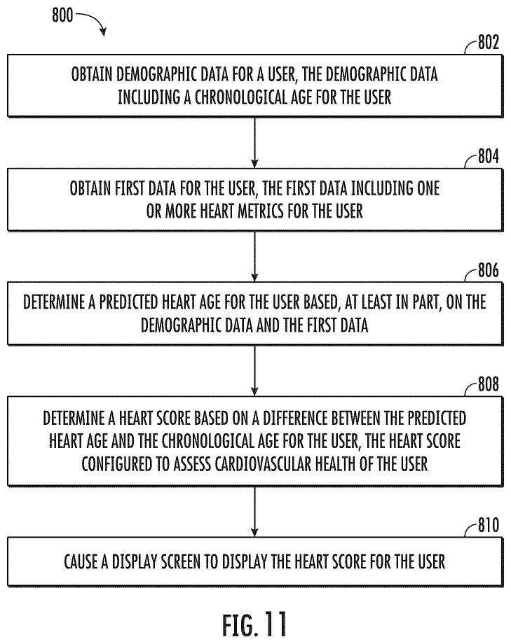

FIG. 11 illustrates a flow diagram of an example computer-implemented method 800 according to one or more example embodiments of the present disclosure. The computer-implemented method 800 can be implemented using, for instance, the wearable computing device 100, the mobile computing device 504, and/or the server system 512 described above with reference to the example embodiments depicted in FIGS. 1-10B The example embodiment illustrated in FIG. 11 depicts operations performed in a particular order for purposes of illustration and discussion. Those of ordinary skill in the art, using the disclosures provided herein, will understand that various operations or steps of the computer-implemented method 800 or any of the other methods disclosed herein can be adapted, modified, rearranged, performed simultaneously, include operations not illustrated, and/or modified in various ways without deviating from the scope of the present disclosure.

As shown at (802), the computer-implemented method 800 may include obtaining, by a computing device (e.g., the wearable computing device 100, the mobile computing device 504, and/or the server system 512) operatively coupled to one or more processors (e.g., the processor(s) 181, the processor(s) 518), demographic data of the user 10. As mentioned, the demographic data can include an age the user 10. The demographic data may be input by the user 10 to the computing device (e.g., via an HMI), as discussed above.

As shown at (804), the computer-implemented method 800 may include obtaining, by a computing device (e.g., the wearable computing device 100, the mobile computing device 504, and/or the server system 512) operatively coupled to one or more processors (e.g., the processor(s) 181, the processor(s) 518), physiological data of the user 10. As mentioned, the physiological data includes one or more cardiac metrics 608 of the user 10. The physiological data may be obtained via one or more physiological sensors 143 embedded and/or integrated in the wearable computing device 100, as discussed above.

As shown at (806), the computer-implemented method 800 may include determining, by a computing device (e.g., the wearable computing device 100, the mobile computing device 504, and/or the server system 512) operatively coupled to one or more processors (e.g., the processor(s) 181, the processor(s) 518), a predicted cardiac age 704 for the user using the demographic data and the physiological data as inputs to a model 702 (as described above).

As shown at (808), the computer-implemented method 800 may include determining, by a computing device (e.g., the wearable computing device 100, the mobile computing device 504, and/or the server system 512) operatively coupled to one or more processors (e.g., the processor(s) 181, the processor(s) 518), a cardiac score based on a difference between the predicted cardiac age and the age for the user. The cardiac score (CScore) can be configured to assess cardiac health of the user 10.

As shown at (810), the computer-implemented method 800 may include causing, by a computing device (e.g., the wearable computing device 100, the mobile computing device 504, and/or the server system 512) operatively coupled to one or more processors (e.g., the processor(s) 181, the processor(s) 518), a display screen of an electronic device (e.g., the wearable computing device 100, the mobile computing device 504, etc.) to display the cardiac score (CScore) for the user 10.

### Additional Disclosure

The technology discussed herein makes reference to servers, databases, software applications, and other computer-based systems, as well as actions performed by, and information sent to and from such systems. The inherent flexibility of computer-based systems allows for a great variety of possible configurations, combinations, and divisions of tasks and functionality between and among components. For instance, processes discussed herein can be implemented using a single device or component or multiple devices or components working in combination. Databases and applications can be implemented on a single system or distributed across multiple systems. Distributed components can operate sequentially or in parallel.

While the present subject matter has been described in detail with respect to various specific example embodiments thereof, each example is provided by way of explanation, not limitation of the disclosure. Those skilled in the art, upon attaining an understanding of the foregoing, can readily produce alterations to, variations of, and equivalents to such embodiments. Accordingly, the subject disclosure does not preclude inclusion of such modifications, variations and/or additions to the present subject matter as would be readily apparent to one of ordinary skill in the art. For instance, features illustrated or described as part of an embodiment can be used with another embodiment to yield a still further embodiment. Thus, it is intended that the present disclosure covers such alterations, variations, and equivalents.

1. A computing device, comprising:
one or more processors; and
one or more computer-readable media that collectively store instructions that, when executed by the one or more processors, cause the computing device to perform operations, the operations comprising:
obtaining demographic data of a user, the demographic data comprising an age for the user;
obtaining physiological data of the user from one or more physiological sensors, the physiological data comprising one or more cardiac metrics of the user;
inputting the demographic data and the physiological data into a machine-learned model configured to output a predicted cardiac age for the user using at least the demographic data and the physiological data as model inputs;
generating a cardiac score based on the predicted cardiac age output by the machine-learned model and the age for the user, the cardiac score configured to assess cardiac health of the user;
obtaining at least one of historical activity data for the user and one or more activity preferences for the user;
determining a recommended activity for the user based on the cardiac score and the at least one of the historical activity data for the user and the one or more activity preferences for the user, wherein the recommended activity is configured to influence the cardiac score of the user to achieve a desired cardiac score;
prompting the user to perform the recommended activity in response to a trigger;
determining an updated cardiac score based on inputting the demographic data and updated physiological data of the user to the machine-learned model, wherein the updated physiological data is obtained from the one or more physiological sensors while the user performs the recommended activity; and
causing a display screen of the computing device to display a comparison of the cardiac score and the updated cardiac score so as to indicate to the user an influence of the recommended activity on the one or more cardiac metrics and the cardiac score.

2. (canceled)

3. (canceled)

4. (canceled)

5. The computing device of claim 1, wherein the operations further comprise:
obtaining anthropometric data for the user; and
inputting the anthropometric data into the machine-learned model, the machine-learned model configured to output the predicted cardiac age for the user using the demographic data, the physiological data, and the anthropometric data as model inputs.

6. The computing device of claim 1, wherein the operations further comprise:
categorizing the cardiac score for the user based, at least in part, on the demographic data for the user; and
causing the display screen to display the categorization.

7. (canceled)

8. The computing device of claim 1, wherein the operations further comprise:
inputting the cardiac score and the physiological data to a second model as second model inputs;
generating, via the second model, one or more explanations providing context for the cardiac score given the physiological data using the second model inputs, the second model being a machine-learned model; and
causing the display screen to display the one or more explanations output by the second model.

9. The computing device of claim 1, wherein the one or more cardiac metrics comprise at least one of a resting heart rate, an average heart rate, a maximum heart rate, a heart rate recovery, and a maximal oxygen consumption.

10. The computing device of claim 1, wherein the computing device is a wearable computing device worn by the user or a mobile computing device.

11. A computer-implemented method for determining a cardiac score for a user, the computer-implemented method comprising:
obtaining, via an electronic device, demographic data of a user, the demographic data comprising an age for the user;
obtaining, via the electronic device, physiological data of the user from one or more physiological sensors of the electronic device, the physiological data comprising one or more cardiac metrics of the user;
inputting, via the electronic device, the demographic data and the physiological data into a machine-learned model of the electronic device, the machine-learned model configured to output a predicted cardiac age for the user using at least the demographic data and the physiological data as model inputs;
generating, via the electronic device, a cardiac score based on the predicted cardiac age output by the machine-learned model and the age for the user, the cardiac score configured to assess cardiac health of the user;
obtaining, via the electronic device, at least one of historical activity data for the user and one or more activity preferences for the user;
determining, via the electronic device, a recommended activity for the user based on the cardiac score and the at least one of the historical activity data for the user and the one or more activity preferences for the user, wherein the recommended activity is configured to influence the cardiac score of the user to achieve a desired cardiac score of the user;
prompting, via the electronic device, the user to perform the recommended activity in response to a trigger;
determining, via the electronic device, an updated cardiac score based on inputting the demographic data and updated physiological data of the user to the machine-learned model, wherein the updated physiological data is obtained via the one or more physiological sensors while the user performs the recommended activity; and
causing a display screen of the electronic device to display a comparison of the cardiac score and the updated cardiac score so as to indicate to the user an influence of the recommended activity on the one or more cardiac metrics and the cardiac score.

12. (canceled)

13. (canceled)

14. (canceled)

15. The computer-implemented method of claim 11, further comprising:
obtaining, via the electronic device, anthropometric data for the user; and
inputting, via the electronic device, the anthropometric data into the machine-learned model, the machine-learned model configured to output the predicted cardiac age for the user using the demographic data, the physiological data, and the anthropometric data as model inputs.

16. The computer-implemented method of claim 11, further comprising:
categorizing, via the electronic device, the cardiac score for the user based, at least in part, on the demographic data for the user; and
causing the display screen to display the categorization.

17. (canceled)

18. The computer-implemented method of claim 11, further comprising:
determining, via a second model of the electronic device, one or more explanations providing context for the cardiac score given the physiological data based on using the cardiac score and the physiological data as second model inputs, the second model being a machine-learned model; and
causing the display screen to display the one or more explanations.

19. The computer-implemented method of claim 11, wherein one or more cardiac metrics comprise at least one of a resting heart rate, an average heart rate, a maximum heart rate, a heart rate recovery, and a maximal oxygen consumption.

20. A computer-implemented method for determining a cardiac score for a user, the computer-implemented method comprising:
obtaining, via an electronic device, demographic data of a user, the demographic data comprising an age for the user;
obtaining, via the electronic device, physiological data of the user from one or more physiological sensors, the physiological data comprising at least one of a resting heart rate, an average heart rate, a maximum heart rate, a heart rate recovery, and a maximal oxygen consumption;
inputting, via the electronic device, the demographic data and the physiological data into a machine-learned model of the electronic device, the machine-learned model configured to output a predicted-cardiac age for the user using at least the demographic data and the physiological data as model inputs;
generating, via the electronic device, a cardiac score based on the predicted cardiac age output by the machine-learned model and the age for the user, the cardiac score configured to assess cardiac health of the user;
inputting, via the electronic device, the cardiac score and the physiological data into a second model of the electronic device as second model inputs;
generating, via the second model, one or more explanations providing context for the cardiac score given the physiological data so as to permit the user to interpret the cardiac score based on using the second model inputs, the second model being a machine-learned model; and
causing a display screen of the electronic device to display the cardiac score for the user and the one or more explanations so as to indicate to the user how the physiological data influences the cardiac score.
